(19) United States
(12) Patent Application Publication
(10) Pub. No.: US 2012/0230574 A1
(43) Pub. Date: Sep. 13, 2012

(54) IMAGING METHOD AND SYSTEM

(75) Inventors: Anatoly Rozenfeld, Redfern (AU); Jan Jakubek, Hyskov (CZ)

(73) Assignee: UNIVERSITY OF WOLLONGONG, North Wollongong, New South Wales (AU)

(21) Appl. No.: 13/498,889

(22) PCT Filed: Sep. 27, 2010
(86) PCT No.: PCT/AU2010/001263

§ 371 Date: May 21, 2012

(30) Foreign Application Priority Data
Sep. 29, 2009 (AU) 2009904772

(51) Int. Cl. G06K 9/00; A61B 8/12; A61B 6/00

(52) U.S. Cl. 382/131; 600/436

(57) ABSTRACT

A probe (14; 14′), comprising an ultrasonic probe (56a,56b; 74a,74b) and a pixellated radiation detector (16) with discrete detecting elements (50a,50b,50c) for detecting a predefined radiation. The probe (14) is adapted to be located at least partially within a body cavity. Also, an imaging method, comprising employing such a probe (14) to form an image while located within a body cavity, and a dosimetry method, comprising employing such a probe (14) to conduct dosimetry while located within a body cavity.

### RELATED APPLICATION

This application is based on and claims the benefit of the filing date of Australian application no. 2009904772 filed 29 Sep. 2009, the content of which as filed is incorporated herein by reference in its entirety.

### FIELD OF THE INVENTION

The present invention relates to an imaging method and system, of particular but by no means exclusive application in the imaging of internal organs such as the prostate.

### BACKGROUND OF THE INVENTION

Prostate cancer is one of the most commonly diagnosed cancers in men over 55 years of age. Approximately 30% of all diagnosed cancers in this age group are prostate carcinomas. Prostate seed brachytherapy is used to treat early-stage, low-risk, prostate cancer and is an alternative to curative prostatectomy in most patients. Brachytherapy can deliver a relatively high radiation dose in a highly conformal fashion to a target site. The highly conformal nature of this treatment allows a significant reduction of dose to the rectum and surrounding structures. The urethra, however, resides within the target volume, so accurate seed placement is critical in maintaining the integrity of a planned dose to this structure.

Low dose rate brachytherapy for early stage disease involves the permanent implantation of radioactive seeds into the prostate. Normally I-125 or Pd-103 seeds are used for prostate seed brachytherapy. These seeds are gamma ray emitters I-125 (E_{γ}˜35.5 keV, and X-ray about 27 keV, T½=59.4 days, initial dose rate about 1 cGy/h at 1 cm distance), Pd-103 (E_{γ}˜21 keV, T½=17.0 days, initial dose rate about 3 cGy/h at 1 cm distance). In comparison with other competing treatment modalities (such as X-rays from a LINAC), I-125 and Pd-103 implantation safely delivers a higher total dose to the target. Another advantage is the short tissue penetration of the gamma photons due to their low energy (with a half layer of 1.3 cm for I-125 and even less for Pd-103), hence sparing surrounding, normal tissue from significant exposure.

Medical complications associated with LDR (low dose rate) brachytherapy treatment of prostate cancer can arise from errors in seed placements during implantation. With random seed placement errors of less than 5 mm, the average dose has been calculated to be 15% lower than prescribed, with a spread of 5-10% (based on random seed displacement). In such cases, simulation has shown that larger dose errors occur.

An existing method for guiding the placement of radioactive seeds is ultrasound image guided transperineal permanent implantation (TPI) for LDR prostate brachytherapy, which is an option for the management of early stage, organ confined prostate cancer [1]. A major drawback of this procedure is that excessive imaging artefacts—produced by the implanted sources—make implant evaluation and modification in real-time unfeasible. CT and MRI guided techniques for prostate brachytherapy have also been developed, and can be performed on patients with large prostates, allowing real-time evaluation and modification of implant geometry [2,3]. However it is impractical and expensive to provide a CT or MRI machine in each operational theatre.

Some efforts have been made using Monte Carlo techniques to develop a pre-planning method that is insensitive to seed misplacement or migration [4,5], though unsuccessfully to date. A 3-D ultrasound seed planning system (SPOT) has become available using 3-D ultrasound imaging and 3-D needle guidance in real-time [6], but this system is expensive, has similar problems with artefacts as does 2-D ultrasound seed imaging and is not able to image individual seed placement (or misplacement) during a treatment procedure.

The Memorial Sloan-Kettering Cancer Center (New York) has developed and successfully implemented intraoperative conformal optimization and planning (I-3D) for ultrasound-based TPI, obviating the need for pre-planning [7]. Software for implementing this approach has been developed using two approaches: generic algorithm and integer programming [8]. This system incorporates acceptable dose ranges allowed within the target as well as dose constraints for the rectal wall and urethra. As part of a pilot study to investigate the feasibility of this approach, 253 patients between 1998 and 2000 and many more subsequently were treated at the Memorial Sloan-Kettering Cancer Center with ultrasound-based I-125 implantation using intraoperative 3-dimensional conformal optimization (I-3D). For the I-3D group, the V100 (percentage of prostate volume receiving 100% of the prescription dose) and D90 (percentage of dose delivered to 90% of the prostate) were 94% and 117%, respectively. The average urethral dose was 140% of the prescription dose. The dosimetric parameters and tolerance profiles were significantly better than in patients treated with a pre-planned implant approach. Among patients treated with the preplanned approach the V100 and D90 were 88% and 95%, respectively. The average urethral dose was 182% of the prescription. The reduced urethral dose associated with the I-3D approach resulted in a significant reduction in acute toxicity during the first year after implantation. The need for alpha-blocker medications to control urinary symptoms (acute grade 2 toxicity) for the first 12 months after the procedure was only 32% for the non-I-3D group and 20% for the I-3D group. Only 2% experienced grade 3 urinary toxicity with this approach. For the non-I-3D group, the incidence of grade 2 urinary symptoms during the first 12 months after the procedure was 58% (p<0.01) [9].

Existing intraoperative dose planning systems can improve the clinical outcome of LDR brachytherapy for prostate cancer treatment if the position of each seed, or each group of seeds, is accurately known in a prostate volume in a suitable frame of reference. In such cases, intraoperative dose planning can be executed in real time based on the known position(s) of the seed(s), comparisons made with a planned dose and compensation made for dosimetry errors through suitable adjustment in the placement of the next seed(s), keeping within real time dose constraints of critical, adjacent organs (e.g. urethra, rectum and bladder) during the seed implantation.

For example, U.S. Pat. No. 7,361,134 discloses a method for determining the seed position in real time based on spectroscopy dosimetry of X-rays from seeds, using three or more radiation mini-detectors [10] installed in the prostate during seed implantation. The mini-detectors can be located within an ultrasound image and seed position relative to the mini-detectors is determined from signals from three or more of these detectors. However, accurate fusion of the ultrasound image and seed location (whether derived with the mini-detectors or, post-operatively, with a fluoroscope) is complicated by the independence of the imaging techniques or apparatus.

One existing technique [13] employs trans-rectal ultrasound-coupled near-infrared optical tomography of the prostate to identify lesions within the prostate in an ultrasound image dataset. This technique provides fused near-infrared and ultrasound images of the prostate using a single transrectal ultrasound probe with built in near-infrared detector.

### SUMMARY OF THE INVENTION

According to a first broad aspect, the present invention provides a probe, comprising:
an ultrasonic probe (that is, having an ultrasonic emitter and ultrasonic detector); and
a pixellated radiation detector with discrete detecting elements for detecting a predefined radiation;
wherein said probe is adapted to be located at least partially within a body cavity, such as a rectum or a vagina.

In a particular embodiment, the probe comprises a shield (typically of a high Z material, such as W or Pb) with at least one window for admitting said radiation (even if with some attenuation) so that the radiation impinges upon said detecting elements generally only if admitted through the at least one window.

It will be appreciated that this need not require that the shield fully surround the detecting elements; the radiation will generally irradiate the detecting elements from a known direction, so the shield will generally only extend between the detecting elements and the expected or known source direction origin of that irradiation.

The shield may be removable.

In one embodiment, the probe comprises a shield with at least one window for admitting said radiation, wherein the shield is movable relative to the pixellated radiation detector between a first position in which the radiation impinges upon the detecting elements generally only if admitted through the at least one window, and a second position in which the shield does not substantially impede the radiation from impinging upon the detecting elements.

Thus, in some applications, the pinhole effect provided by the window(s) may not be required, so the shield would be rotated, retracted or otherwise removed from impeding the radiation from impinging upon the detecting elements.

For example, the shield may rotatable within a housing of the probe between the first position (essentially over the detecting elements) and the second position (essentially under, for example, the detecting elements). In such an embodiment, the shield may be semi-cylindrical (or at least arcuate in cross section).

In another example, the shield may be mounted to be retractable within a housing of the probe between the first position (essentially over the detecting elements) and the second position (retracted from the detecting elements).

It will be understood that, depending on the nature of the radiation, a small amount of the radiation may reach the detecting elements without passing through the window (as the material of the shield may not completely block the radiation), but that generally this will be at such a low level that counts arising from the detecting elements can be attributed to radiation admitted through the window.

In many embodiments, the ultrasonic probe and the pixellated radiation detector are located in fixed relative position, but in some embodiments this relative position may be adjustable (but generally fixed during use).

In an embodiment, the pixellated radiation detector comprises a plurality of individual radiation detectors.

In one embodiment, the pixellated radiation detector comprises one or more semiconductor pixellated radiation detectors (such as Medipix (trade mark) detectors).

In certain embodiments, the pixellated radiation detector comprises a plurality of individual radiation detectors of at least two different types or of at least two different energy responses.

Thus, the pixellated radiation detector might comprise, for example, a first individual radiation detector adapted for the higher detection efficiency of 511 keV photons (such as a pixellated CdTe detector) and a second individual radiation detector adapted for the higher detection efficiency of 20 to 40 keV photons (such as a pixellated Si detector).

In certain embodiments, the shield has a plurality of said windows. In these embodiments, the windows may be arranged relative to the pixellated detector such that at least some (or in some embodiments, all) of the detecting elements of the pixellated detector receive radiation admitted through only one of said windows.

This can be achieved by, for example, providing the probe with an internal wall or walls about the window or windows (or between adjacent windows) that prevent the radiation from impinging upon other than a predefined set (or respective sets) of detecting elements.

The internal wall or walls may be of, for example, the same material as the shield.

In one embodiment, the probe has a housing that comprises the shield.

In another embodiment, the housing comprises a plastic wall that is generally transparent to the radiation, wherein the shield is located within said housing.

In one embodiment, the ultrasonic probe and the pixellated radiation detector are arranged for imaging overlapping volumes.

In another embodiment, the probe is adapted to be rotated or translated to bring the ultrasonic probe and the pixellated radiation successively into position for imaging a specified volume.

According to a second broad aspect, the present invention provides an imaging system, comprising the probe described above.

In one embodiment, the system includes an image fusion module for fusing an image from the pixellated radiation detector and an ultrasound image from the ultrasonic probe.

In a particular embodiment, the system includes a drive for rotating the probe between a first position for collection of data with the ultrasonic probe and a second position for detecting the predefined radiation.

In an embodiment, the system includes a radiation source, wherein the probe is adapted to detect photons from the source, to scan the source relative to the pixeliated detector, and to generate an image (such as a CT image, or a fluoroscopic image).

The radiation source may comprise, for example, an X-ray source (such as comprising one or more X-ray tubes), a low energy gamma-ray emitting radioactive source, or multiple individual sources (including a mixture of types of source).

In another embodiment, the pixellated radiation detector is adapted to detect 511 keV gamma rays, the system includes a further imaging detector adapted to detect 511 keV gamma rays and a coincidence discriminator in data communication with the pixellated radiation detector and the further imaging detector, and the system is configured to perform PET imaging.

According to a third broad aspect, the present invention provides an imaging method, comprising:
employing the probe described above to form an image while located within a body cavity, such as a rectum or a vagina.

According to a fourth broad aspect, the present invention provides a dosimetry method, comprising:
employing the probe described above to conduct dosimetry while located within a body cavity, such as a rectum or a vagina.

In one embodiment, the method includes determining dose or dose rate at the probe. In another embodiment, the method includes determining dose or dose rate at an adjacent tissue from dose or dose rate at the probe.

This may be used during, for example, LDR brachytherapy (such as to monitor the radiation dose received by adjacent organs or tissues).

It should be noted that any of the various features of each of the above aspects of the invention can be combined as suitable and desired.

### BRIEF DESCRIPTION OF THE DRAWING

In order that the invention may be more clearly ascertained, embodiments will now be described, by way of example, with reference to the accompanying drawing, in which:

FIG. 1 is a schematic view of an imaging system according to an embodiment of the present invention;

FIG. 2 is a schematic view of a robotic mount with rectal probe (with integrated pixellated radiation detector) of the imaging system of FIG. 1;

FIG. 3 is a photograph of the rectal probe of the imaging system of FIG. 1;

FIG. 4 is a schematic, partial cut-away view of the rectal probe of the system of FIG. 1, showing the pixellated radiation detector of the rectal probe;

FIG. 5 is another schematic, partial cut-away view of the rectal probe of the system of FIG. 1, showing the pixellated radiation detector of the rectal probe;

FIGS. 6A and 6B are schematic elevational and plan views of the rectal probe of the system of FIG. 1;

FIGS. 7A and 7B are schematic elevational and plan views of a variant of the rectal probe of the system of FIG. 1;

FIG. 8A and 8B are schematic, partial cut-away view of the rectal probe of the system of FIG. 1 in use with a subject;

FIG. 9 is a schematic view of the probe of the system of FIG. 1 in use with a subject;

FIG. 10 is a photograph of a prostate PMMA phantom with seeds with lead foil pinhole above a Medipix detector according to the embodiment of FIG. 1;

FIGS. 11A and 11B are positive and negative views of the lead foil pinhole of FIG. 10;

FIGS. 12A, 12B and 12C are images (shown in negative for clarity) from the setup of FIG. 10 of respectively one, two and three 0.8×4 mm I-125 seeds in the prostate PMMA phantom, with an acquisition time of 1 to 2 s;

FIG. 13 is a schematic view of an imaging system according to a second embodiment of the present invention for use in a CT mode or in fluoroscopy mode; and

FIG. 14 is a schematic view illustrating the operation of the imaging system of FIG. 13;

FIG. 15 is a schematic view of an experimental arrangement according to the embodiment of FIG. 13;

FIG. 16 is an image (shown in negative for clarity) from the arrangement of FIG. 15 of dummy seeds in a prostate phantom; and

FIG. 17 is a schematic view of the probe of an imaging system according to a fifth embodiment of the present invention in use with a subject.

### DETAILED DESCRIPTION

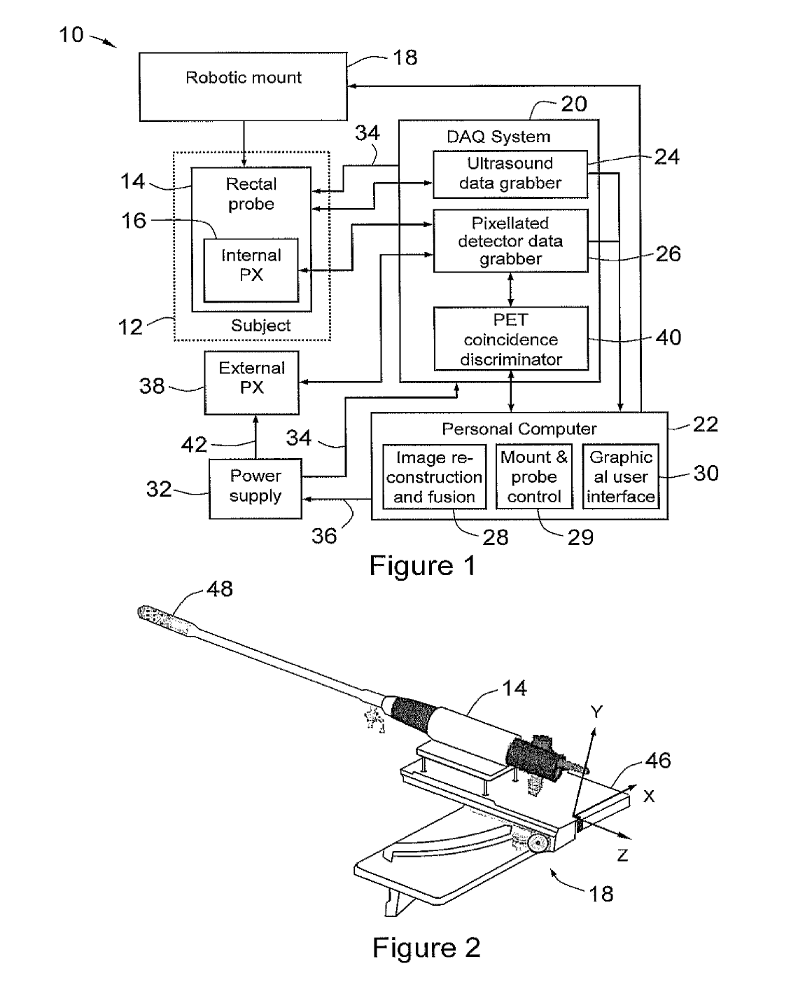

An imaging system according to an embodiment of the present invention is shown at 10 in FIG. 1, together with a subject to be imaged 12.

Imaging system 10 includes an imaging probe in the form of rectal probe 14 with an integrated pixellated radiation detector 16. Pixellated radiation detector 16 is referred to in what follows as “internal” pixellated radiation detector 16 because, in use, it is intended to be located inside the rectum (or other cavities) of subject 12, though it will be appreciated by those skilled in the art that it can also be used in other externally.

Rectal probe 14 is, in general, a transrectal ultrasound (TRUS) probe, with end-fire and longitudinal ultrasonic emitters/receivers, adapted for ultrasound image guided transrectal or transperineal imaging (such as of the type provided by Aloka Co., Ltd of Japan with distributed sagittal transducers along its axis or by Brüel & Kjær Sound and Vibration Measurement A/S of Denmark). However, rectal probe 14 incorporates—as explained above—internal pixellated radiation detector 16, as is described in greater detail below. Imaging system 10 includes a robotic mount 18 for supporting and guiding rectal probe 14, a data acquisition (DAQ) system 20 for acquiring image data from rectal probe 14 and internal pixellated detector 16, and a personal computer 22 for receiving, reconstructing and fusing image data from DAQ system 20 and for controlling imaging system 10.

DAQ system 20 includes an ultrasound data grabber 24 for receiving ultrasound data from rectal probe 14, and a pixellated detector data grabber 26 for receiving data from internal pixellated detector 16 via a digital bus. The outputs of both ultrasound data grabber 24 and pixellated detector data grabber 26 are connected to personal computer 22.

Personal computer 22 has an image reconstruction and fusion module 28, a mount and probe control module 29 and a graphical user interface 30. Image reconstruction and fusion module 28 receives data from ultrasound data grabber 24 and pixellated detector data grabber 26, transforms that data to construct images, determines the locations of radiation sources (such as radioactive seeds) and fuses ultrasonic images with radiation source images. Mount and probe control module 29 is adapted to control the position and orientation of robotic mount 18 and hence of rectal probe 14. Graphical user interface 30 is operable by a user to control imaging system 10, including image reconstruction and fusion module 28 and mount and probe control module 29.

Imaging system 10 also includes a power supply 32 for supplying power to DAQ system 20 and, via DAQ system 20, to rectal probe 14 (including internal pixellated detector 16), via digital power link 34. A slow control data link 36 is also provided between personal computer 22 and power supply 32, so that power supply 32 can be controlled from personal computer 22.

Another embodiment of the invention is comparable to imaging system 10 but additionally includes an external pixellated detector 38 for PET imaging. This embodiment is discussed further below.

Referring to FIG. 2, rectal probe 14, as discussed above, is typically deployed in the rectum for ultrasonically imaging (in particular the prostate), so robotic mount 18 includes an x-y-z stage 46, controllable by mount and probe control module 29 from personal computer 22, for locating the sensitive volume 48 of rectal probe 14 in the rectum of subject 12.

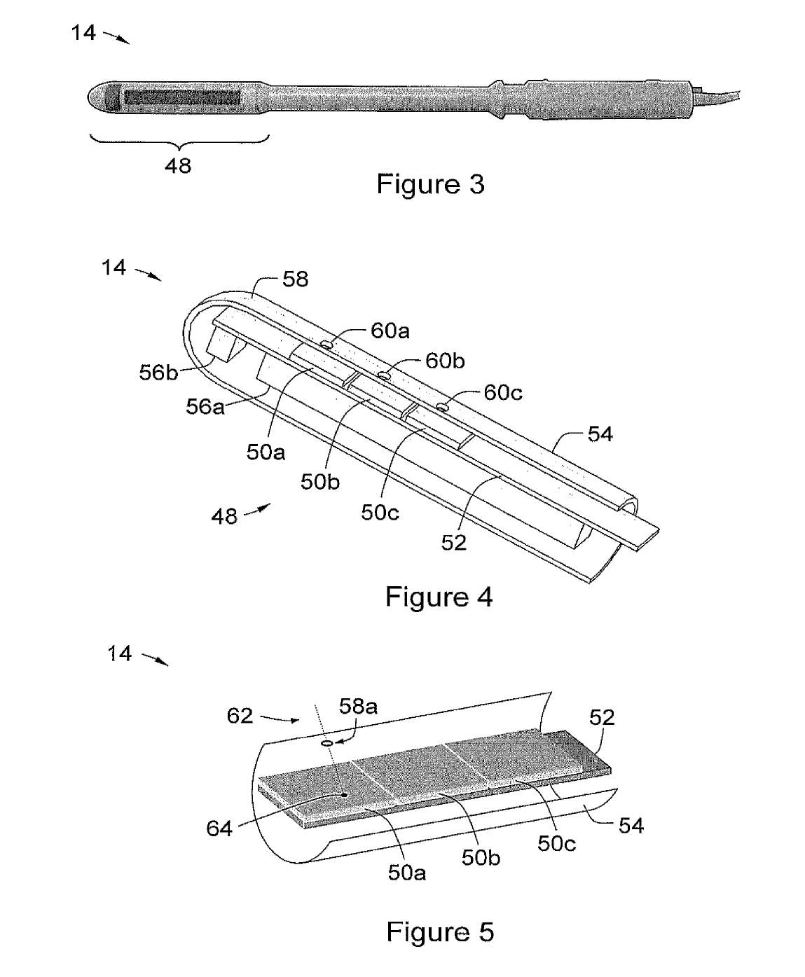

FIG. 3 is a photograph of rectal probe 14 removed from robotic mount 18, indicating sensitive volume 48, while FIG. 4 is a schematic cut-away view of the sensitive volume 48 of rectal probe 14. As discussed above, rectal probe 14 includes an integrated pixellated detector in the form of internal pixellated detector 16. Pixellated detector 16 comprises one or more semiconductor pixellated detectors, in this embodiment in the form of three Medipix (trade mark) detectors 50a, 50b, 50c. Medipix detectors 50a, 50b, 50c are mounted on a kapton board 52.

Medipix detectors 50a, 50b, 50c are high spatial resolution pixellated silicon detectors each with a sensitive area of 15×15 mm² and 56,000 independent pixels (each of size 50×50 μm²). Each of Medipix detectors 50a, 50b, 50c has a read-out chip (not shown) on its back face. The detecting elements of Medipix detectors 50a, 50b, 50c are separated from the associated electronics owing to the confined space within probe 14.

It will be appreciated that, while in this embodiment internal pixellated radiation detector 16 has three Medipix detectors 50a, 50b, 50c, in other embodiments a single Medipix detector may be sufficient or desirable (such as if a more compact probe is required), while other embodiments may have two Medipix detectors or, indeed, more than four or more Medipix detectors.

Furthermore, while imaging system 10 includes a rectal probe 14 with an integrated pixellated detector 16, in some embodiments according to the present invention the internal pixellated detector 16 may be located in a dedicated probe (i.e. without ultrasonic probe functionality), or in a probe that includes some other form of detector rather than a ultrasonic probe.

Medipix detectors 50a, 50b, 50c are provided in internal pixellated detector 16 because they are compact and—being pixellated—can be used to obtain spatially resolved data. Other detectors with comparable properties may be employed in alternative embodiments of imaging system 10.

Rectal probe 14 has an outer housing 54 of plastics material that provides rectal probe 14 with structural integrity and, being generally water-tight, protects its functional components. Rectal probe 14 also includes a shield (not shown) within the outer housing that is (in this embodiment) generally semi- or part-cylindrical and of a high Z material such as tungsten or lead, and located over Medipix detectors 50a, 50b, 50c. In this embodiment, the shield comprises a 1 mm thick tungsten foil (i.e. sufficient to attenuate essentially all 27 keV radiation from I-125 seeds), above Medipix detectors 50a, 50b, 50c and immediately within and conforming to cylindrical outer housing 54 (to which it is fastened).

As a result, the shield is essentially opaque to the radiation (generally in the form of gamma rays or X-rays of around 20 to 40 keV) to be detected by internal pixellated radiation detector 16. Rectal probe 14 also has a sagittal ultrasonic transducer 56a and a transverse ultrasonic transducer 56b, located on the underside (with respect to Medipix detectors 50a, 50b, 50c) of kapton board 52. Sagittal ultrasonic transducer 56a is located essentially opposite Medipix detectors 50a, 50b, 50c, while transverse ultrasonic transducer 56b is located in forward tip 58 of rectal probe 14. Housing 54 may include ultrasonic windows adjacent to sagittal ultrasonic transducer 56a and transverse ultrasonic transducer 56b of a material that attenuates the ultrasonic waves less than does the plastics material of the rest of housing 54.

In order to admit the radiation from the radiation sources to internal pixellated radiation detector 16, the shield has three pinhole windows 60a, 60b, 60c, each located over and—in this embodiment—centred on a respective Medipix detector 50a, 50b, 50c. (In another embodiment, each pinhole window 60a, 60b, 60c is located over the respective Medipix detector 50a, 50b, 50c, but not centred on the respective Medipix detector 50a, 50b, 50c. Such an offset may be advantageous in some applications, such as according to the geometry of the intended use.)

In FIG. 4, pinhole windows 60a, 60b, 60c are shown as though provided in housing 54 in order to suggest their location, but in practice they are provided in the shield and would not be visible from outside rectal probe 14; the radiation detected by Medipix detector 50a, 50b, 50c is not greatly attenuated by the plastics material of housing 54.

Pinhole windows 60a, 60b, 60c admit the radiation and are arranged so that radiation admitted through a specific one of pinhole windows 60a, 60b, 60c can only impinge the corresponding one of Medipix detectors 50a, 50b, 50c. (If necessary, a wall of shield material can be located between each adjacent pair of pinhole windows (60a,60b; 60b,60c), extending from the shield to kapton board 52 or the plane of Medipix detectors 50a, 50b, 50c. The perpendicular distance from each of pinhole windows 60a, 60b, 60c to the detecting elements of Medipix detectors 50a, 50b, 50c is 6 to 7 mm.

The location at which that radiation impinges (or equivalently the specific detecting element that detects the radiation) is a function of the origin of the radiation, so the direction from which that radiation has been received can be determined from the location of that individual detecting element.

The high spatial resolution of the Medipix detectors 50a, 50b, 50c (viz. 50 μm) means that the distance between the pinhole windows 60a, 60b, 60c and the plane of Medipix detectors 50a, 50b, 50c need not be large while still allowing a detailed image of the seed in the plane of pixellated radiation detector 16 to be obtained, and still allowing the location of the seed in three dimensions relative to the detector to be determined, by image reconstruction and fusion module 28. (This may be compared to existing CT scanners or pinhole gamma cameras, in which imaging detectors have low radial spatial resolution and large magnification is required, so a large distance is required between the imaged object and the detector array.)

FIG. 5 is schematic partial cut-away view of the sensitive volume 48 of rectal probe 14 illustrating the function of pinhole windows 60a, 60b, 60c. When radiation 62 arrives from a radioactive source, it is admitted to rectal probe 14 only if it passes through one of the pinhole windows, in this example forward pinhole window 60a. Radiation 62 impinges the corresponding Medipix detector (in this example forward Medipix detector 50a), giving rise to a detection event in the pixel 64 at the intersection of the gamma ray's path and the Medipix detector. The location of that pixel is correlated with the direction from which the radiation 62 was received.

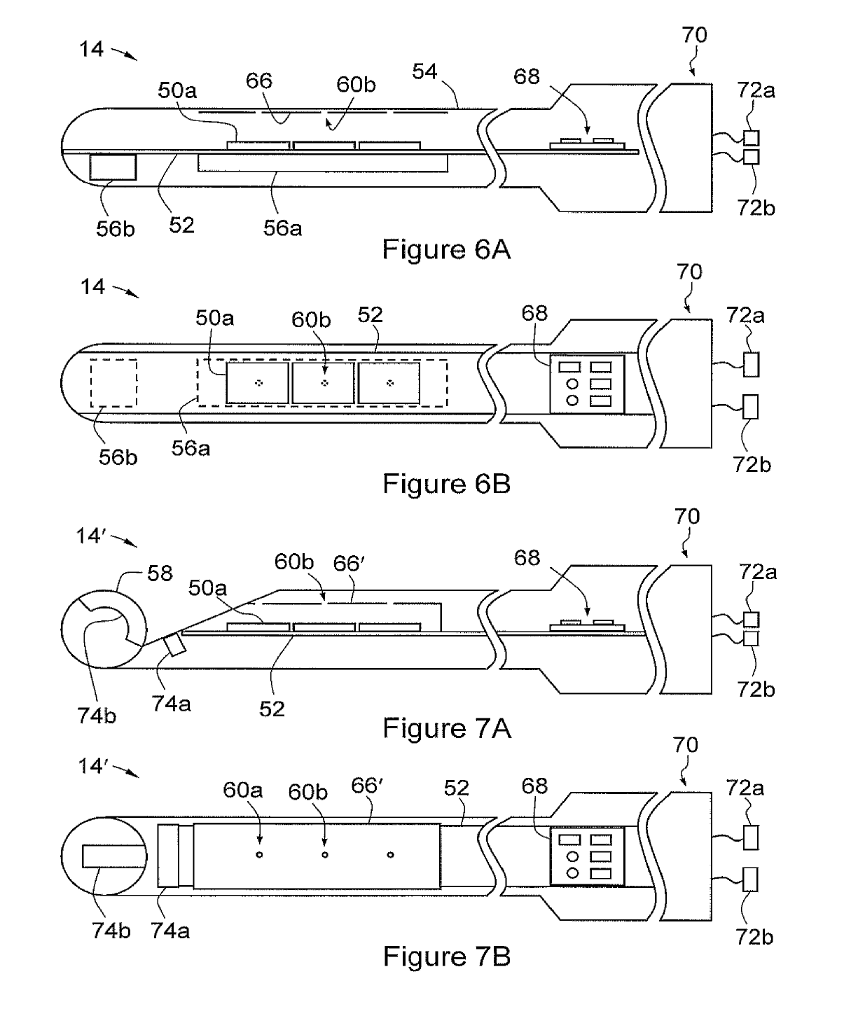

FIGS. 6A and 6B are schematic elevational and plan views of rectal probe 14 according to this embodiment. FIG. 6A indicates the location of shield 66 under outer housing 54 and above Medipix detectors 50a, 50b, 50c. Readout electronics 68 for Medipix detectors 50a, 50b, 50c are located in the tail 70 of rectal probe 14. Medipix output signals are output from a first USB connector 72a, while ultrasonic transducer signals (from sagittal ultrasonic transducer 56a and transverse ultrasonic transducer 56b) are output from a second USB connector 72b; both USB connectors 72a, 72b extend out of tail 70 of rectal probe 14.

In use, rectal probe 14—once appropriately located—is used to collect radiation image data with Medipix detectors 50a, 50b, 50c then, under control of mount and probe control module 29, rotated about its long axis by 180°. Ultrasound image data is then collected with sagittal ultrasonic transducer 56a and transverse ultrasonic transducer 56b. Both datasets are transmitted to image re-construction and fusion module 28 for processing.

FIGS. 7A and 7B are schematic elevational and plan views of rectal probe 14′ according to one variant of this embodiment. Rectal probe 14′ is similar to rectal probe 14 of FIGS. 6A and 6B, but—instead of having sagittal ultrasonic transducer 56a and transverse ultrasonic transducer 56b—rectal probe 14′ has an axial ultrasonic transducer 74a (for traverse prostate imaging) forward of kapton board 52 and a sagittal ultrasonic transducer 74b (for longitudinal prostate imaging) in forward tip 58 of rectal probe 14′.

FIG. 7A also indicates the location of shield 66′ (which includes a rear wall and hence is somewhat different from shield 66 of FIG. 6A), located under outer housing 54 and above Medipix detectors 50a, 50b, 50c.

In use, rectal probe 14′—once appropriately located—is used to collect radiation image data (with Medipix detectors 50a, 50b, 50c) and ultrasound image data (with sagittal ultrasonic transducer 56a and transverse ultrasonic transducer 56b) essentially simultaneously. Both datasets are transmitted to image re-construction and fusion module 28 for processing.

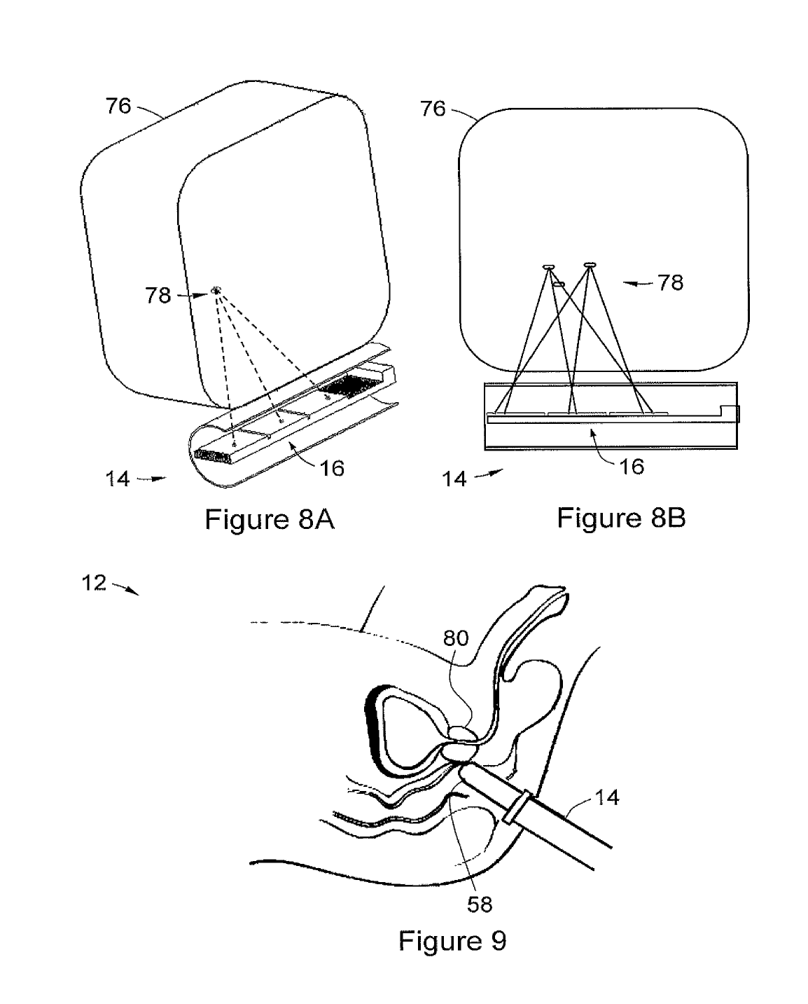

Most radiation sources of the type being considered here emit radiation isotropically, so a single source (or seed) should be detected—in this embodiment—by all three Medipix detectors 50a, 50b, 50c. FIGS. 8A and 8B are respectively perspective and elevation schematic views of this situation, depicting a notional object 76 to be imaged (which may be an organ such as the prostate). One or more I-125 seeds 78 are located in object 76, each giving rise to detection events in—as illustrated—Medipix detectors 50a, 50b, 50c, and permitting each seed 78 to be imaged. Image reconstruction and fusion module 28 uses the data from internal pixellated detector 16 to construct an image, and determine the three-dimensional location, of each seed 78 within prostate.

Image reconstruction and fusion module 28 determines the three-dimensional location of each seed based on data from one or more of Medipix detectors 50a, 50b, 50c. The direction of each seed 78 is apparent from the location of the detecting elements that capture the radiation from the respective seed 78. Image reconstruction and fusion module 28 determines the distance between a respective seed 78 and those detecting elements from the size of the image and the known size (and shape) of the seeds 78. This is facilitated, in this embodiment, by the high resolution of Medipix detectors 50a, 50b, 50c, which provide accurate projection images of seeds 78.

If a particular seed 78 is imaged by more than one of Medipix detectors 50a, 50b, 50c, its three-dimensional location is determined by image reconstruction and fusion module 28 a corresponding number of times and averaged, thereby providing a still more accurate location.

Thus, image reconstruction and fusion module 28 determines the three-dimensional locations of seeds 78 relative to Medipix detectors 50a, 50b, 50c, then fuses these seed locations with an ultrasonic image generated by rectal probe 14 at essentially the same time, and outputs the resulting, fused image to the screen of personal computer 22.

Imaging system 10 can thus be used to facilitate, for example, precise seed positioning, with the newly implanted seed's location being tracked almost in real time. Indeed, the high efficiency of X-ray registration of Medipix detectors 50a, 50b, 50c (due in large part to the thickness—about 0.3 mm—of the Medipix silicon detectors that make up Medipix detectors 50a, 50b, 50c), satisfactory count statistics can be recorded, for 50 micron resolution, in 1 s (or less in some cases).

It should also be noted that Medipix detectors 50a, 50b, 50c permit parallel independent readout of each pixel (hence 56,000 readout channels for each detector), which provides a large dynamic range of measured X-ray intensities, and hence the ability of imaging radioactive sources (e.g. seeds) that are very close to Medipix detectors 50a, 50b, 50c. This is especially advantageous when seeds are placed in the lower (i.e. posterior) part of the prostate close to the rectum (and hence to rectal probe 14). The small size of Medipix detectors 50a, 50b, 50c facilitates the close mutual proximity of Medipix detectors 50a, 50b, 50c within rectal probe 14, and their close placement—once rectal probe 14 is in situ—to the organ of interest.

FIG. 9 is a schematic view of a male subject 12 into whose rectum rectal probe 14 has been inserted. Forward tip 58 of rectal probe 14 is located as closely as possible to the prostate 80 of subject 12. Once rectal probe 14 is thus located, radioactive seeds may be inserted sequentially into prostate 80, with the prostate and each dropped seed imaged with imaging system 10 so that the appropriate location of the successive seed can be determined. It is expected that seed position in a prostate could thus be determined to a high accuracy (of, in this embodiment, about 0.1 mm).

Imaging system 10 thus has a number of advantages over existing techniques, providing direct three-dimensional real-time in vivo seed imaging in an US image dataset in LDR for each dropped seed. Spectroscopic techniques, such as are described in U.S. Pat. No. 7,361,134, allow position measurements to be made based on dose rate measurements from the seed, but only provide average position. CT scanner guided implantation provides additional external irradiation to the patient, is very expensive and is unavailable in most theatres.

In addition, no external X-ray source is required, unlike in CT guided imaging, and imaging system 10 is composed of relatively inexpensive components. Imagine fusion is straightforward, as seed position need only be placed on the simultaneously collected US image data set.

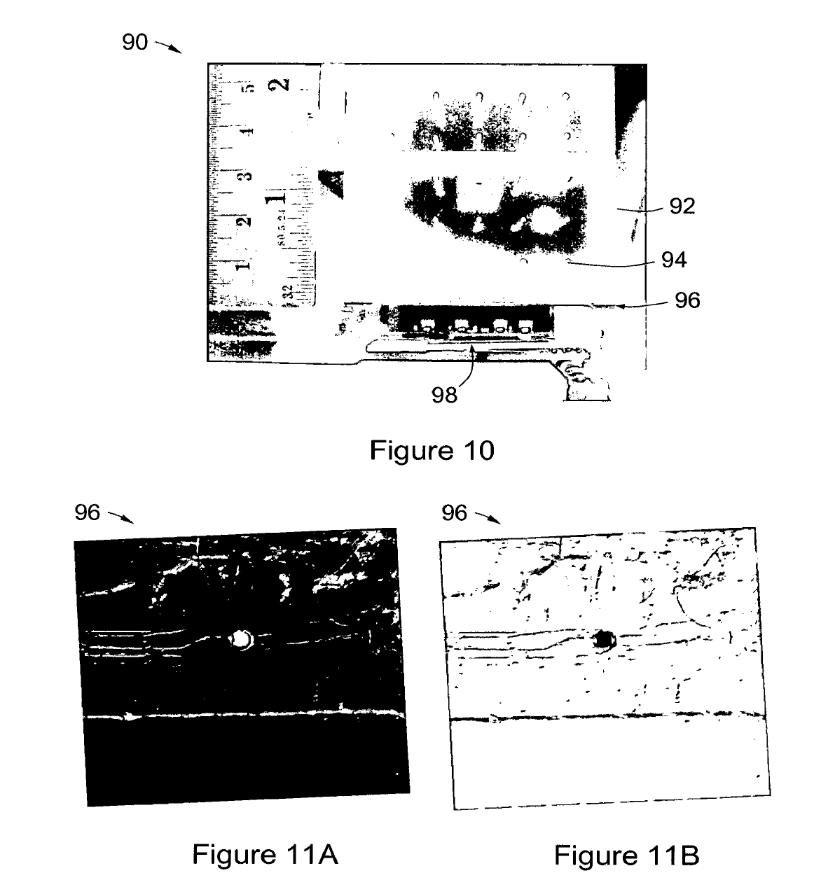

Referring to FIG. 10, in order to test the inventive concept embodied in imagining system 10, a prostate phantom 92 of PMMA (polymethylmethacrylate) was constructed and provided with a plurality of slots 94 for receiving I-125 radioactive seeds (of 0.8×4 mm). A lead foil 96 (seen edge-on in this view) with a pinhole was located immediately below prostate phantom 92; a Medipix detector 98 was located about 7 mm below lead foil 96.

FIGS. 11A and 11B are views (FIG. 11B in negative for clarity) of lead foil 96. The pinhole has a diameter of 0.5 mm.

Images of a single seed (located low in prostate phantom 92), two seeds (low and centred in prostate phantom 92) and three seeds (low, centred and high in prostate phantom 92) were collected, in each case for 1 to 2 seconds.

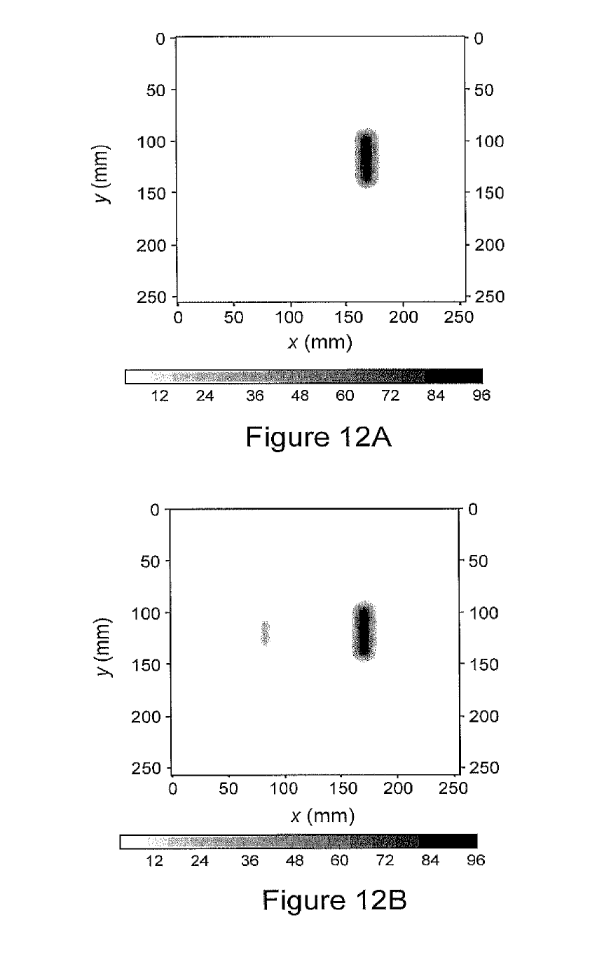

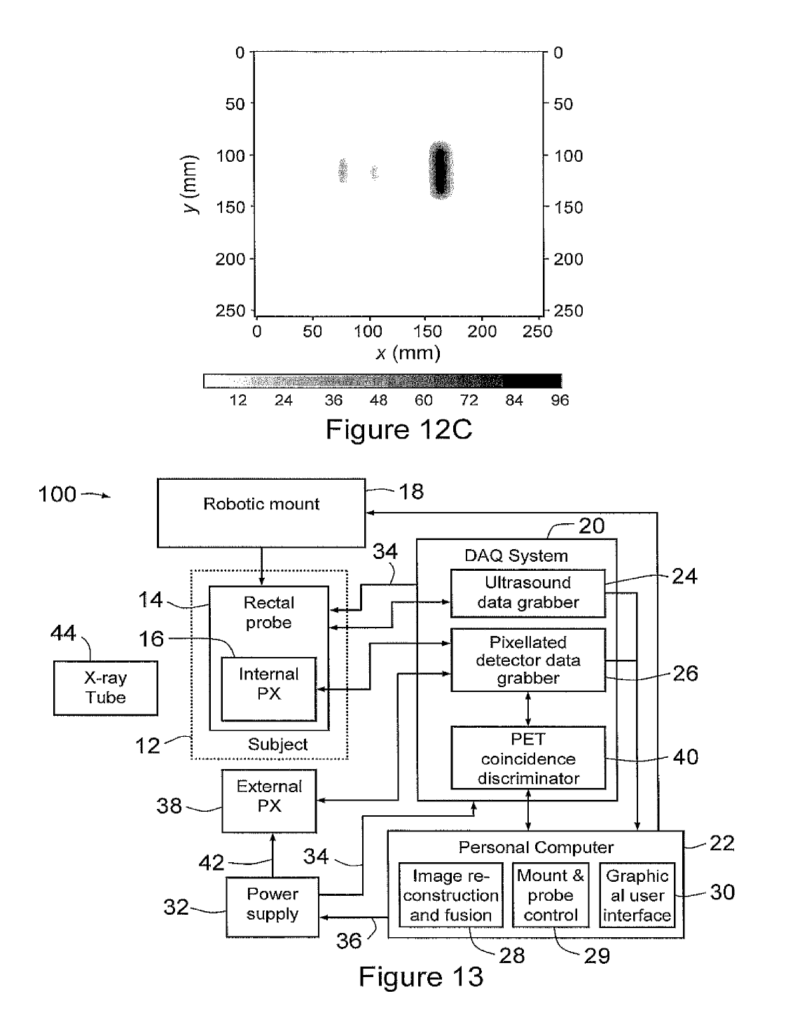

FIGS. 12A, 12B and 12C are the respective, resulting images (in negative for clarity). Even though no optically focusing elements have been employed, the seeds have nonetheless been imaged satisfactorily and are clearly resolvable.

FIG. 13 is a schematic view of an imaging system 100 according to a second embodiment of the present invention. Imaging system 100 is similar to imaging system 10, and like reference numerals have been used to identify like features. In addition, imaging system 100 has an X-ray source, in this embodiment in the form of an X-ray tube 44 (though other X-ray sources may also be employed). X-ray tube 44 may be referred to as an external X-ray source because, in use, it is located outside subject 12. Furthermore, unlike shield 66 of imaging probe 14 of FIG. 1, in this embodiment the shield is either semi-cylindrical and rotatable within the housing of imaging probe 14 or removable therefrom, so as to fully expose Medipix detectors 50a, 50b, 50c to radiation, as—in this embodiment—the pinhole effect of windows 60a, 60b, 60c is not required.

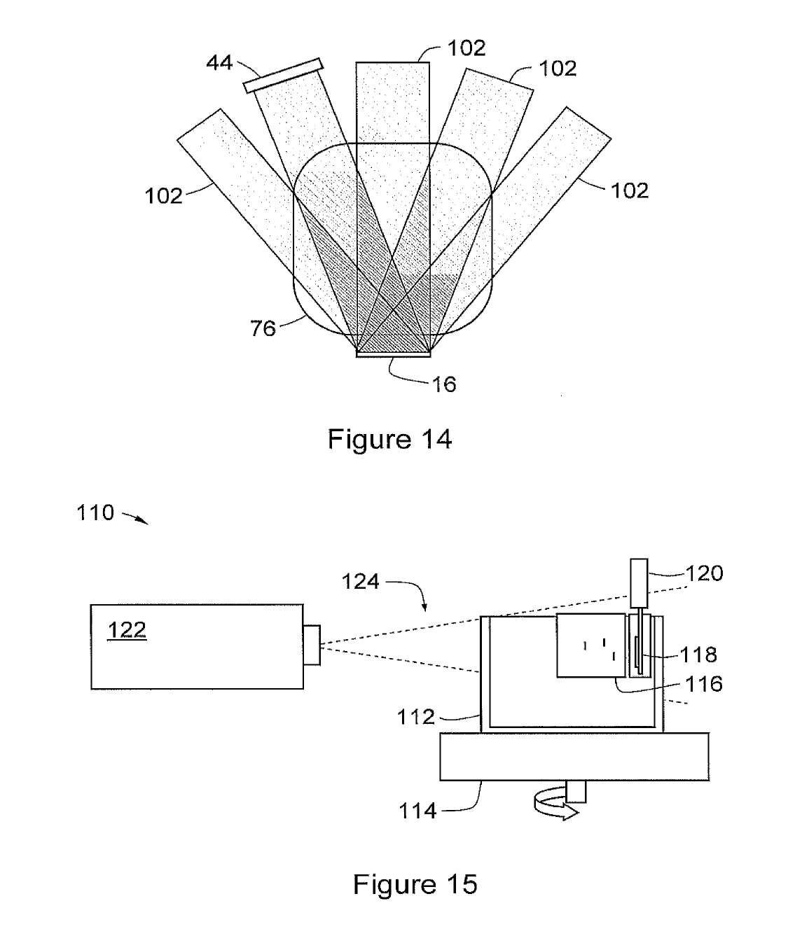

Consequently, imaging system 100 can produce a high spatial resolution CT scan or fluoroscopic image of, in this example, the prostate or seeds in the prostate. This is done by locating rectal probe 14 in the rectum close to the prostate and moving X-ray tube 44 in an arc (of, in this example, about 90° with a gantry (not shown, also controlled from personal computer 22) over the subject's pelvis, while continually collecting a sequence of image data. This is illustrated in FIG. 14, which depicts the prostate 76 (whether before implantation or with implanted seeds not shown) above internal pixellated detector 16, and with X-ray tube 44 at one point in its arc. The directions of successive X-ray illumination are shown at 102, at each of which locations image data is collected. The prostate image or seeds' locations are then reconstructed using a known CT or fluoroscopy limited-angle algorithm in image reconstruction and fusion module 28.

The prostate 76 or seeds therein are thus readily imaged. In particular, the seeds are of high Z material, such as silver or titanium, so produce excellent contrast with low X-ray exposure.

This embodiment has several advantages, including higher spatial resolution than conventional CT scanners (as used in this application) owing to the proximity of internal pixellated detector 16 to the prostate and lower photon requirement for obtaining high contrast images (owing to the high pixilation of the detector). In addition, CT imaging of the prostate can be performed with imaging system 100 in the operating theatre immediately prior to seed implantation or HDR brachytherapy, with rectal probe 14 (including an internal pixellated detector 16) in situ, obviating any need to change configuration or position of the prostate or of the detectors (thereby also maximizing resolution).

Furthermore, existing CT guided imaging systems employing CT scans are expensive and rarely available in operating theatres.

The inventive concept of imaging system 100 in fluoroscopy mode was tested using an experimental arrangement 110 shown schematically in FIG. 15. An intensity modulated radiation therapy (IMRT) torso phantom 112 was placed on a rotating table 114, with a prostate phantom 116 (provided with dummy, non-radioactive seeds of size 0.8×4 mm) located within IMRT torso phantom 112; a Medipix detector 118 (with USB connector 120) was located immediately behind prostate phantom 116 (as it would be in, for example, a rectum).

An orthovoltage 50 kV X-ray tube 122 was located beside rotating table 114 at a height that would irradiate prostate phantom 116 and Medipix detector 118 with a horizontal X-ray beam 124 successively from different angles.

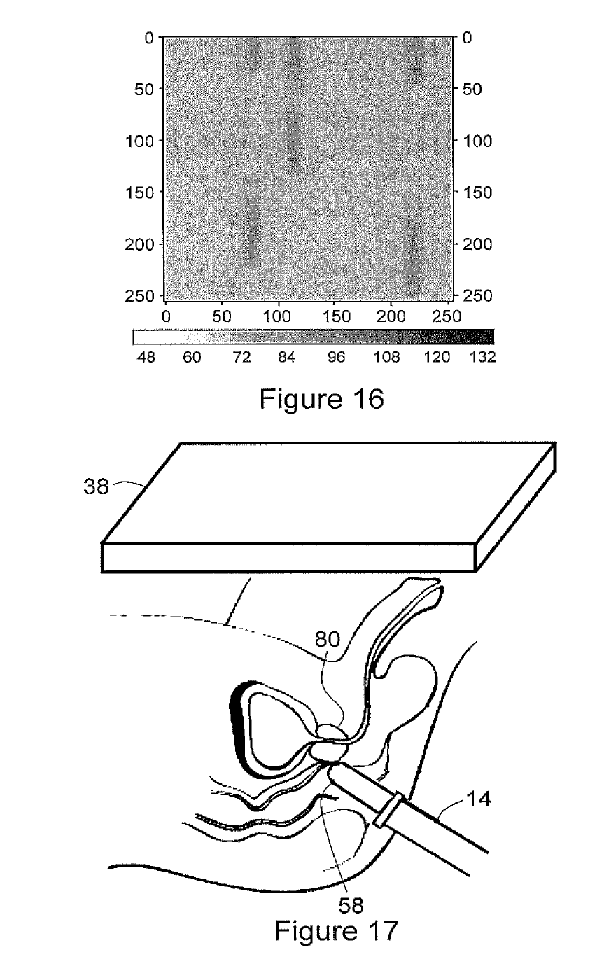

FIG. 16 is an image obtained with experimental arrangement 110 at an irradiation angle of 10° (being the angle between the axis of IMRT torso phantom 112 and the direction of the X-ray beam, controlled by rotating table 114). By obtaining images of the seeds' projections on the detector plane of Medipix detector 118 at different angles allows one to obtain three dimensional seed position in, for example, a prostate after implantation. That is, post implant dosimetry can thus be performed.

The quality of experimental arrangement 110 can be judged by the clarity of the Ti shells of the dummy seeds, and of the voids (of 0.4×0.6 mm²) visible in each dummy seed where the radioactive (e.g. I-125) material would otherwise be located.

A dose monitoring system according to a third embodiment of the present invention is comparable to imaging system 10 of FIG. 1. However, rather than being used for imaging (or exclusively for imaging), this embodiment is used for monitoring or measuring the radiation dose deposited by the radioactive seeds (or other radiation source) at a radiation sensitive location (such as at the wall of an organ or of the rectum), particularly in conjunction with LDR brachytherapy. A two-dimensional dose image collected with Medipix detectors 50a, 50b, 50c operating in spectroscopy mode (such as is as described in U.S. Pat. No. 7,361,134) is indicative of dose in the plane of the Medipix detectors 50a, 50b, 50c and indeed can be recalculated to the surface of rectal probe 14 (being close to the plane of Medipix detectors 50a, 50b, 50c) and hence at the surface of the material surrounding rectal probe 14 (viz. the rectum).

Thus, in this embodiment (as in system 100 of FIG. 13), the shield is either semi-cylindrical and rotatable within the housing of imaging probe 14 or simply removable therefrom, so as to fully expose Medipix detectors 50a, 50b, 50c to radiation, as—in this embodiment—the pinhole effect of windows 60a, 60b, 60c is not required.

Personal computer 22 of this embodiment is adapted to convert data from Medipix detectors 50a, 50b, 50c to dose at the surface of the surrounding material and, hence, in medical applications, of surrounding tissue (such as rectum wall). The dose monitoring system of this embodiment can thus act as, for example, a quality assurance tool for use during LDR brachytherapy, to monitor the radiation dose received by adjacent organs or tissues.

The dose monitoring system of this embodiment has several advantages. Existing techniques do not provide comparable spatial resolution or, if they do (such as in the case of GAF film), without providing real-time quality assurance.

It should be understood that this embodiment may be provided in imaging system 10 of FIG. 1. The resulting imaging and dose monitoring system can be used both to image one or more radioactive seeds in an ultrasound dataset image and to monitor the radiation dose of tissues adjacent to probe 14 (such as the wall of the rectum) during or following seed implantation. Both can thus be done without disturbing instrumentation (such as by rotating shield 66 through 180° when dosimetry mode is required) or the subject.

A source tracking system according to a fourth embodiment of the present invention is also comparable to imaging system 10 of FIG. 1 or imaging system 100 of FIG. 13. However, rather than being used exclusively for imaging, this embodiment is adapted for tracking the position or activity of a radioactive seed (such as 10 Ci activity Ir-192 seed) while in the subject for high dose rate (HDR) brachytherapy, particularly of the prostate.

The source tracking system of this embodiment thus generates images in the same manner as does system 10 of FIG. 1 or system 100 of FIG. 13, but for a single movable high activity Ir-192 (or other radioisotope) seed. The system locates the position of the seed as it is inserted into the prostate (in this example) by means of the data outputted by internal pixellated detector 16 of rectal probe 14.

In variations of this embodiment, the tracking is performed using an immediate TRUS, CT, MRI or other image of the prostate. The system displays the evolving location of the seed against that existing image and, using a position tracking and comparison module of the system, compares that position with the planned or intended location of the seed at any particular moment. This thus permits quality assurance of the HDR brachytherapy to be performed.

The system of this embodiment has a number of advantages, including being compact, ‘in body’, high resolution and real-time. Being ‘in body’ means that the detector (viz. internal pixellated detector 16 is essentially fixed in location relative to, in this example, the prostate and close to the sources being tracked. In addition, internal pixellated detector 16 has higher resolution than is typical of existing approaches. For example, Duan et al. [12] propose a fluorescent screen-based pinhole camera, but the properties of such a system (e.g. low spatial resolution of fluorescent screens) make in body deployment impossible.

Furthermore, the operation of the system of this embodiment functions is independent of tissue equivalency or homogeneity of the medium (such as the prostate) through which the source is moving.

An imaging system according to a fifth embodiment of the present invention is also comparable to imaging system 10 of FIG. 1 or imaging system 100 of FIG. 13. However, as foreshadowed above, the imaging system of this embodiment includes an additional pixellated detector 38, which is referred to in what follows as “external” pixellated detector 38 because, in use, it is intended to be located outside subject 12.

External pixellated detector 38 is controlled from personal computer 22; like internal pixellated detector 16, external pixellated detector 38 transmits acquired data to pixellated detector data grabber 26 of DAQ system 20 for forwarding to personal computer 22.

DAQ system 20 of this embodiment also includes a PET coincidence discriminator 40, which is in data communication with pixellated detector data grabber 26 and personal computer 22. In this embodiment, the system includes an analogue low voltage power link 42 from power supply 32 to external pixellated detector 38. Furthermore, in this embodiment the system includes a remotely controllable gantry (not shown) for supporting, guiding and orienting external pixellated detector 38, and a slow control data link between personal computer 22 and the gantry, so that external pixellated detector 38 and the gantry can be controlled from personal computer 22.

According to this embodiment, internal pixellated detector 16 comprises pixellated CdTe (rather than silicon) detectors for higher detection efficiency of 511 KeV gamma rays.

External pixellated detector 38 is of any type suitable for PET coincidence detection. For example, external pixellated detector 38 may be comparable to internal pixellated detector 16 or comprise a pixellated scintillator and PMT.

Referring to FIG. 17, in use, external pixellated detector 38 is located above the pelvis of subject 12 for detecting 511 keV gamma rays generated in positron-electron annihilation events, and forward tip 58 of rectal probe 14 is located as closely as possible to the prostate 80 of subject 12. External pixellated detector 38 is gated for coincidence with a corresponding event in internal pixellated detector 16 by PET coincidence discriminator 40. The resulting data are transmitted to image reconstruction and fusion module 28 of personal computer 22, which generates a three-dimensional image of the distribution of the positron emitting tracer (which will concentrate in the more aggressive cells), and automatically fuses that image with the US or X-ray image produced with US prove 14 or X-ray tube 44.

Modifications within the scope of the invention may be readily effected by those skilled in the art. It is to be understood, therefore, that this invention is not limited to the particular embodiments described by way of example hereinabove.

In the claims that follow and in the preceding description of the invention, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments of the invention.

Further, any reference herein to prior art is not intended to imply that such prior art forms or formed a part of the common general knowledge in Australia or any other country.

### REFERENCES

1. A. T. Porker, J. C. Blasko, P. D. Grimm, S. M. Raddy, H. Ragde, “Brachytherapy for prostate cancer,” Ca. Cancer J. Clin. 45 (1995) 165-178

2. J. A. Molloy, M. B. Williams, “Treatment planning considerations and quality assurance for CT-guided tarnsischiorectal implantation of the prostate,” Med. Phys. 26(9) (1999) 1943

3. D. H. Brinkman, R. W. Kline, “Automated seed location from CT datasets of the prostate,” Med. Phys. 25(9) (1998) 1667

4. R. Taschereau, J. Roy, J. Pouliot, “Monte Carlo simulations of prostate implants to improve dosimeter and compare planning method,” Med. Phys. 26(9) (1999) 1952

5. P. L. Roberson, V. Navayana, D. L. McSchan et al., “Source placement error for permanent implant of the prostate,” Med.Phys. 24(2) (1997) 251

6. “SPOT: 3D Ultrasound Brachytherapy Planning, System for Seed Implants”, Nucletron brochure, 1999

7. Zelefsky M. J., Yamada Y., Cohen G., Venkatraman E. S., Fung A Y, Furhang E, Silvern D, Zaider M., “Post-implantation dosimetric analysis of permanent transperineal prostate implantation: improved dose distributions with an intraoperative computer-optimized conformal planning technique,” Int. J. Radiat. Oncol. Biol. Phys. 48 (2000) 601-8

8. Lee, Zaider M. et al., Phys. Med. Biol. 44 (1999) 145-165

9. Wallner K. E., Roy J., Harrison L. et al., “Dosimetry guidelines to minimize urethral and rectal morbidity following transperineal I-125 prostate brachytherapy”, Int J Radiat Oncol Biol Phys. 32 (1995) 465-471

10. U.S. Pat. No. 7,361,134, A. B. Rozenfeld, M. Zaider “Method and apparatus for real time dosimetry”, issued 22 Apr. 2008

11. U.S. Pat. No. 6,847,838, S. Majewski et al., “Apparatus and method for high dose rate brachytherapy radiation treatment”, issued 25 Jan. 2005

12. J. Duan, D. J. Macey, P. N. Pareek and I. A. Brezovich, “Real-time monitoring and verification of in vivo high dose rate brachytherapy using a pinhole camera,” Med. Phys. 28(2) (2001) 167-173

13. G. Xu, D. Piao, C. H. Musgrove, C. F. Bunting and H. Dehghani, “Trans-rectal ultrasound-coupled near-infrared optical tomography of the prostate,” Parts I and II, Optic Express 6(22) (2008) 17484-17504 & 17505-17520

1. A probe, comprising:
an ultrasonic probe; and
a pixellated radiation detector with discrete detecting elements for detecting a predefined radiation;
wherein said probe is adapted to be located at least partially within a body cavity.

2. A probe as claimed in claim 1, further comprising a shield with at least one window for admitting said radiation so that the radiation impinges upon said detecting elements generally only if admitted through the at least one window.

3. A probe as claimed in claim 2, wherein said shield is removable.

4. A probe as claimed in claim 1, further comprising a shield with at least one window for admitting said radiation, wherein the shield is movable relative to the pixellated radiation detector between a first position in which the radiation impinges upon the detecting elements generally only if admitted through the at least one window, and a second position in which the shield does not substantially impede the radiation from impinging upon the detecting elements.

5. A probe as claimed in claim 4, wherein the shield is rotatable within a housing of the probe between the first position and the second position.

6. A probe as claimed in claim 5, wherein the shield is semi-cylindrical or is arcuate in cross section.

7. A probe as claimed in claim 4, wherein the shield is mounted to be retractable within a housing of the probe between the first position and the second position.

8. A probe as claimed in claim 2, wherein the shield has a plurality of said windows.

9. A probe as claimed in claim 8, wherein the windows are arranged relative to the pixellated detector such that at least some of the detecting elements of the pixellated detector receive radiation admitted through only one of said windows.

10. A probe as claimed in claim 2, wherein said probe includes an internal wall or walls about or between the at least one window that prevent the radiation from impinging upon other than a predefined set or respective sets of detecting elements.

11. A probe as claimed in claim 2, wherein the probe has a housing that comprises the shield.

12. A probe as claimed in claim 2, wherein the housing comprises a wall of plastics material that is generally transparent to the radiation, wherein the shield is located within said housing.

13. A probe as claimed in claim 1, wherein the ultrasonic probe and the pixellated radiation detector are located with adjustable relative positions.

14. A probe as claimed in claim 1, wherein the pixellated detector comprises a plurality of individual radiation detectors.

15. A probe as claimed in claim 1 wherein the pixellated radiation detector comprises one or more semiconductor pixellated radiation detectors.

16. A probe as claimed in claim 15, wherein the pixellated detector comprises one or more Medipix detectors.

17. A probe as claimed in claim 14, wherein the pixellated radiation detector comprises a plurality of individual radiation detectors of at least two different types or of at least two different energy responses.

18. A probe as claimed in claim 1, wherein the ultrasonic probe and the pixellated radiation detector are arranged for imaging overlapping volumes.

19. A probe as claimed in claim 1, wherein the probe is adapted to be rotated or translated to bring the ultrasonic probe and the pixellated radiation successively into position for imaging a specified volume.

20. An imaging system, comprising a probe according to claim 1.

21. A system as claimed in claim 20, further comprising an image fusion module for fusing an image from the pixellated radiation detector and an ultrasound image from the ultrasonic probe.

22. A system as claimed in claim 20, further comprising a drive for rotating the probe between a first position for collection of data with the ultrasonic probe and a second position for detecting the predefined radiation.

23. A system as claimed in claim 20, further comprising a radiation source, wherein the probe is adapted to detect photons from the source, to scan the source relative to the pixellated radiation detector, and to generate an image.

24. A system as claimed in claim 23, wherein the radiation source comprises an X-ray source, a low energy gamma-ray emitting radioactive source, or multiple individual sources.

25. A system as claimed in claim 20, wherein the pixellated radiation detector is adapted to detect 511 keV gamma rays, the system includes a further imaging detector adapted to detect 511 keV gamma rays and a coincidence discriminator in data communication with the pixellated radiation detector and the further imaging detector, and the system is configured to perform PET imaging.

26. An imaging method, comprising:
employing a probe according to claim 1 to form an image while located within a body cavity.

27. A dosimetry method, comprising:
employing a probe according to claim 1 to conduct dosimetry while located within a body cavity.

28. A method as claimed in claim 27, including determining dose or dose rate at the probe.

29. A method as claimed in claim 27, including determining dose or dose rate at an adjacent tissue from dose or dose rate at the probe.
